(19) United States
(12) Patent Application Publication
(10) Pub. No.: US 2007/0259023 A1
(43) Pub. Date: Nov. 8, 2007

(54) Insulin compensated nutrition

(76) Inventor: Josh N. Hogan, Los Altos, CA (US)

Correspondence Address: Josh Hogan, 620 Kingswood Way, Los Altos, CA, 94022, US

(21) Appl. No.: 11/789,351

(22) Filed: Apr. 23, 2007

Related U.S. Application Data
(60) Provisional application No. 60/797,935, filed on May 5, 2006.

(51) Int. Cl. A61K 38/28; A61K 9/50; A61K 47/00

(52) U.S. Cl. 424/439; 424/490; 514/3

(57) ABSTRACT

A product and method of producing a product and a system are taught usefully in providing nutrition whose glucose content is compensated for by embedded insulin containing capsules. The invention includes techniques to protect the insulin in some environments and which release the insulin in other environments to form nutrient which has a reduced impact on the blood glucose concentration when ingested. Such protective techniques include encapsulating insulin in pH-sensitive hydro-gels which protect insulin in low ph environments and expand to release the insulin in higher pH environments. Additional stabilizing coatings can enhance long term storage of the insulin compensated nutrient.

### CROSS REFERENCES TO RELATED APPLICATIONS

This utility patent application JH070421US claims priority from provisional application No. 60/797,935 (docket number JH060504P), filed on 5 May 2006 the entirety of which is incorporated by reference as if fully set forth herein.

### FIELD OF INVENTION

This invention relates to control of blood glucose concentration levels in diabetic patients. In particular it relates to techniques and methods for reducing the impact of nutrition on the blood glucose concentration levels of diabetics when ingested. It involves techniques for protecting and releasing insulin in a pre-determined manner in different environments.

### BACKGROUND OF THE INVENTION

Individuals with diabetes have an impaired ability to automatically control their blood glucose levels. This typically requires them to monitor their blood glucose levels regularly and frequently self-administer insulin, currently typically by subcutaneous injection, in order to maintain their blood glucose levels at acceptable levels. Blood glucose levels of diabetics are particularly prone to large fluctuations after nutrition or food has been taken.

The conventional method of monitoring blood glucose levels involves drawing blood and measuring the level with a disposable test strip and a monitoring device. This is typically painful, inconvenient, costly and has serious impact on the diabetic individual's quality of life. Implantable and in-dwelling glucose monitors are available and although less painful, they are also inconvenient, costly and have serious impact on the diabetic individual's quality of life.

The painful and inconvenient aspects associated with conventional blood glucose monitoring and insulin delivery frequently lead to poor compliance and inaccurate control of the blood glucose level. Other insulin delivery mechanisms are under development, including: pulmonary; buccal (through inner cheek wall) and ingested (eaten or swallowed). Insulin is typically rendered unusable by the acidic environment of the stomach, however the insulin may be protected from the acidic environment by being encapsulated in a protective hydro-gel.

Failure to appropriately control blood glucose levels can cause irreversible cumulative organ damage which can have dire health and quality of life consequences to the diabetic individual. Such dire health consequences can include, kidney disease, limb amputations, blindness, dental disease, nervous system disease, blood pressure and fatal heart disease. There are also considerable economic costs associated with the consequences of improperly controlled blood glucose levels. In light of the considerations described above there is an unmet need to reduce blood glucose level fluctuations and thereby improve blood glucose level control.

### SUMMARY OF THE INVENTION

The invention provides a method for creating a foodstuff product and inventive product by virtue of the inventive process, where such product is useful for providing nutrition whose glucose content is compensated for by embedded insulin containing capsules. The invention includes techniques which protect the insulin in some environments and which release the insulin in other environments to form nutrient which has a reduced impact on the blood glucose concentration when ingested. Such protective techniques include encapsulating insulin in pH-sensitive hydro-gels which protect insulin in low ph environments and expand to release the insulin in higher pH environments. Additional stabilizing coatings can enhance long term storage of the insulin compensated nutrient. The invention also a system for controlling or regulating glucose levels and calibrating glucose monitors.

### BRIEF DESCRIPTION OF THE DRAWINGS

FIG. 1 is an illustration of a preferred embodiment of the inventive product.

FIG. 2 is an illustration of typical operation of the preferred embodiment.

FIG. 3 is an illustration of a system for controlling glucose levels.

FIG. 4 illustrates generally a method for producing the product of FIG. 1.

### DETAILED DESCRIPTION OF THE INVENTION

Typical food substances, referred to herein as nutrients, result in blood glucose levels increasing when ingested by individuals (including humans and animals). Healthy individuals automatically control blood glucose levels by a combination of internally making insulin or glucose available within the individual. In diabetic individuals the ability to control blood glucose levels is impaired and can lead to inappropriate blood glucose levels with dire health, cost, and quality of life consequences.

For purposes of this invention, nutrients (or nutrition substances) include, but are not limited to, foodstuffs in general; solid food substances; liquid food substances, beverages, soups, etc.; chewable substances; dietary supplements, and in general substances that are typically ingested by being eaten, drunk, chewed or sucked, irrespective of the value of the nutrition provided.

Insulin is now readily available in various forms including liquid, and aerosol form. The liquid form is typically used by diabetics for subcutaneous injection to reduce blood glucose levels. The aerosol form includes glucose in small particle form and is used by diabetics for pulmonary delivery. Digesting insulin directly in the same manner as nutrition is digested is problematic because the insulin will encounter an acidic environment in the stomach region which will render the insulin useless.

Small particles of insulin can be encapsulated in hydro-gels which can protect the insulin when in the acidic environment of the stomach. The hydro-gel can be designed to be pH sensitive such that when the insulin containing hydro-gel is in a strongly acidic or low pH environment it encapsulates the insulin in protective manner, but when the hydro-gel is in a less acidic or higher pH environment, such as in the intestine tract, it expands which relaxes its encapsulation and makes the insulin available for absorption into the blood stream.

If such pH sensitive insulin containing hydro-gel capsules are distributed or embedded throughout a nutrition substance in a concentration related to the glucose content of the nutrition substance, then when the nutrition substance is digested (by eating, drinking or chewing it) a corresponding amount of insulin will also be ingested. Ideally, the resulting amount of insulin released into the blood stream and the timing of that release will compensate for the increased blood glucose level caused by digesting the nutrition substance. Ideally the released insulin will balance the digested glucose.

The insulin containing hydro-gel capsules may also have a protective coating that keeps the capsules stable while embedded in the nutrition substance. The protective coating is designed to dissolve in a specific environment, such as one with a specific pH range, and optionally to dissolve with a controlled time constant. The protective coating enables stable storage of the capsule embedded nutrition. Such nutrition with embedded insulin is herein referred to as insulin compensated nutrition.

In the preferred embodiment of this invention, a food, or nutrient, is evaluated to determine its glucose content either by analysis or by reviewing published or otherwise available information relating to its glucose content. The quantity of insulin necessary to compensate for the glucose that will be delivered into the blood by digesting the nutrient is determined.

This compensating quantity determination may be made by analysis of the nutrient, comparison with similar accumulated information or by monitoring the blood glucose level after digesting similar nutrients. This determination may also involve the efficiency with which the insulin will be absorbed into the blood stream. This efficiency factor can be determined by means that include continuous or frequent glucose level monitoring and analysis.

Insulin particles are encapsulated in pH sensitive hydro-gels. The hydro-gels are selected to form a protective coating around the insulin particles which remains protective in an acidic or low pH environment. The hydro-gels are designed to cease to be protective in a higher pH environment which allows the insulin to become available.

The hydro-gel property that protects the insulin can include mechanical properties, which tightly encapsulates insulin particles in an acidic environment and expands or relaxes such encapsulation in a less acidic environment. Tight encapsulation prevents interaction with the insulin particles. Relaxed encapsulation allows the insulin particles to separate from the hydro-gel and to interact with other substances.

The insulin containing hydro-gel capsules can optionally be further protected by applying a further protective coating to the hydro-gel capsules. This coating is designed to reduce or eliminate any interaction between the hydro-gel capsules and the particular nutrient into which they may be embedded. This coating can be designed to dissolve in a low pH environment which then allows the pH sensitive nature of the hydro-gel capsules to determine release of the insulin particles.

For purposes of this invention such pH sensitive hydro-gels capsules encapsulating insulin particles in a protected manner in some environments and releasing the insulin in another environment are referred to as reversibly protected insulin components. Additionally coated pH sensitive hydro-gel capsules containing insulin, with such additional coatings soluble in some environments are also referred to as reversibly protected insulin components. Other approaches for protecting insulin in some environments and making the insulin available in other environments are also referred to as reversibly protected insulin components.

The reversibly protected insulin components are embedded in the nutrient in a manner that is substantially randomly distributed. The amount of the reversibly protected insulin components is such that it will substantially compensate for the amount of glucose in the nutrient or in the nutrient and associated nutrients, such as in the case where the nutrient with embedded insulin would also be used to substantially compensate for the glucose content of a conventional meal. Nutrient with reversibly protected insulin components that substantially compensates for the glucose content of the nutrient (or the nutrient and associated nutrients) is herein referred to as insulin compensated nutrient.

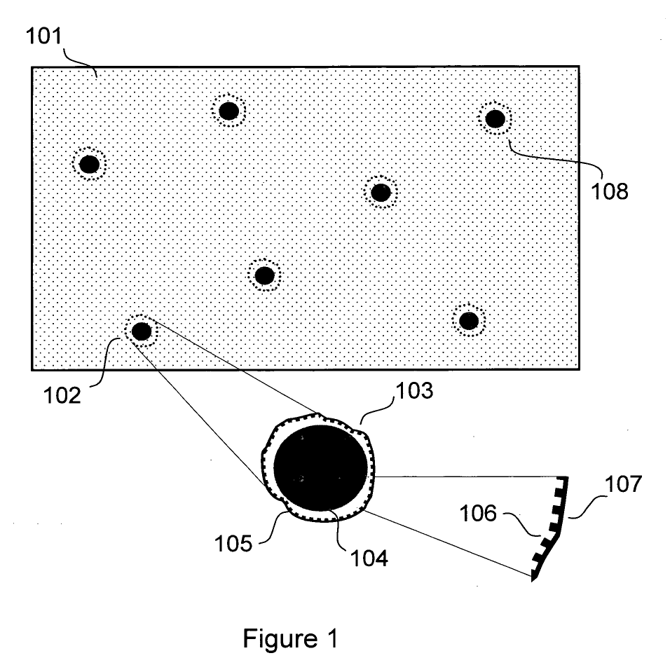

The preferred embodiment is illustrated in and described with respect to FIG. 1 where a nutrient 101 has multiple distributed insulin containing capsules. One of the capsules 102 is shown in an enlarged version 103 which shows it containing an insulin particle 104 within a protective hydro-gel 105. In a further enlarged section, the surface of the hydro-gel 106 has a coating 107 that maintains the capsules in a stable condition when embedded in the nutrient 101.

In the preferred embodiment, the coating 107 protects the capsules, such as 108, when embedded in the nutrient 101 by preventing interaction between the nutrient and the hydro-gel and thereby maintaining it in a stable condition. The coating dissolves or is rendered useless when it encounters an acidic environment and thus is a reversible protective coating.

The hydro-gel 105 encapsulates the insulin particle 104 in a protective manner when in a low pH (or strongly acidic) environment, however when it encounters a higher pH (or less acidic) environment the hydro-gel expands and allows the insulin particle 104 to diffuse or become available for interaction. The hydro-gel thus has a reversibly protective attribute. The nutrient 101 with an appropriate amount of embedded reversibly protected insulin constitutes insulin compensated nutrient.

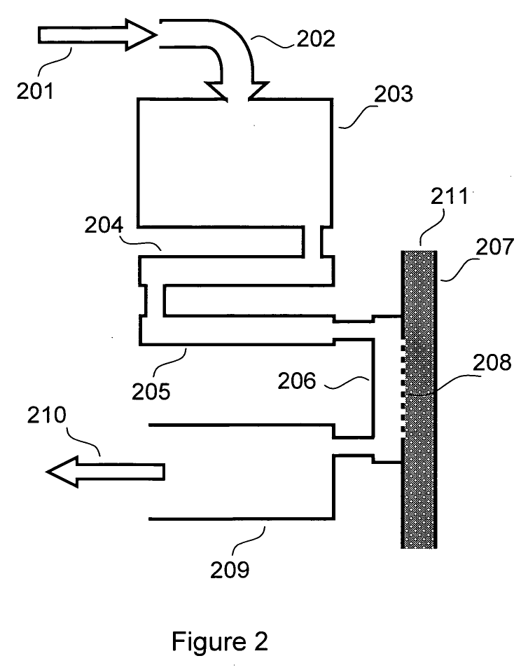

Operation of the insulin compensated nutrient is illustrated in and described with respect to FIG. 2 where the insulin compensated nutrient 201 is taken orally and delivered via the throat 202 to a stomach 203. The environment of a stomach is very acidic, having a low pH value of the order of 1.2 typically. This low pH environment dissolves the optional protective coating on the insulin containing hydro-gel capsules. However the hydro-gel capsules tightly bind the insulin particles, preventing them from interacting with the environment, thus protecting them.

The nutrient and insulin containing hydro-gel capsules pass from the stomach 203 to the compartments of the small intestine, consisting of the duodenum 204 then the jejunum 205, then the ileum 206 and then into the large intestine 209 (beginning with the cecum). The partially or fully digested nutrient 210 continues to travel through the large intestine to be further processed in the normal manner.

The environment of the ileum 206 has a higher pH value of the order of 6.8 typically. The pH sensitive hydro-gel capsules no longer tightly bind the insulin particles in this higher pH or very weakly acidic environment allowing the insulin particles to separate from the hydro-gels and to interact with the environment. The blood stream 211 in blood carrying vessels, such as indicated by 207, can interact with the ileum 206 typically through a membrane indicated by the dashed line 208. For purposes of this invention, the arrangement described above, and similar arrangements, are referred to as a digestive complex.

The reversibly protected insulin, or that is now unprotected insulin, passes through the membrane 208 and is absorbed into the blood stream 211, thus delivering the insulin to the blood stream and making the insulin available to substantially compensate for glucose from the nutrient. Timely compensation for ingested insulin reduces the magnitude of glucose concentration level excursions and thereby provides improved control of the blood glucose concentration level.

The degree of ph-sensitivity of the hydro-gels can control the timing of release of the insulin and its absorption into the blood stream. Controlling the statistical distribution of the degree of ph-sensitivity of the hydro-gels can control the temporal profile of release of the insulin and its absorption into the blood stream. This statistical distribution can be selected to optimize operation of specific insulin compensation nutrients. Specific nutrients can also be selected to control the timing of delivery of the insulin to the blood stream.

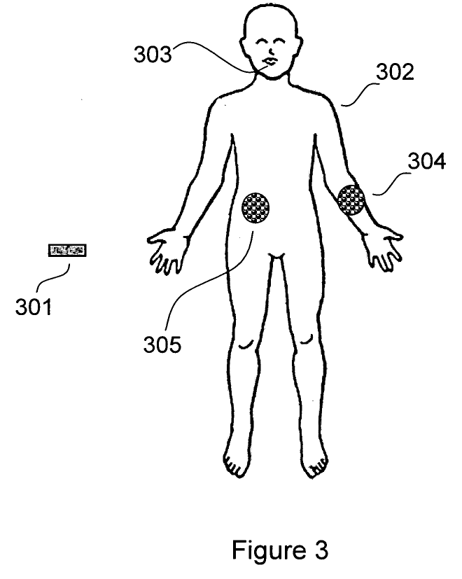

FIG. 3 illustrates the overall glucose control system which is comprised of a unit of insulin compensated nutrient 301, a human 302 which includes a digestive complex. The insulin compensated nutrient 301 is ingested through the mouth 303 and the insulin is delivered via the digestive complex to the blood stream. A non-invasive glucose monitor 304 measures glucose levels and can also generate calibration data from the measured glucose levels resulting from the ingested insulin compensated nutrient. Alternatively or additionally an indwelling glucose monitor 305 measures glucose levels and can also generate calibration data from the measured glucose levels resulting from the ingested insulin compensated nutrient. Such calibration data can be used to calibrate the glucose monitor being used and may also be made available, for example over the internet, to be combined with other calibration data and used more generally. Although the system depicted applies to humans, the system has other application, including but not limited to, veterinary applications that can be appreciated by those of skill in the relevant arts.

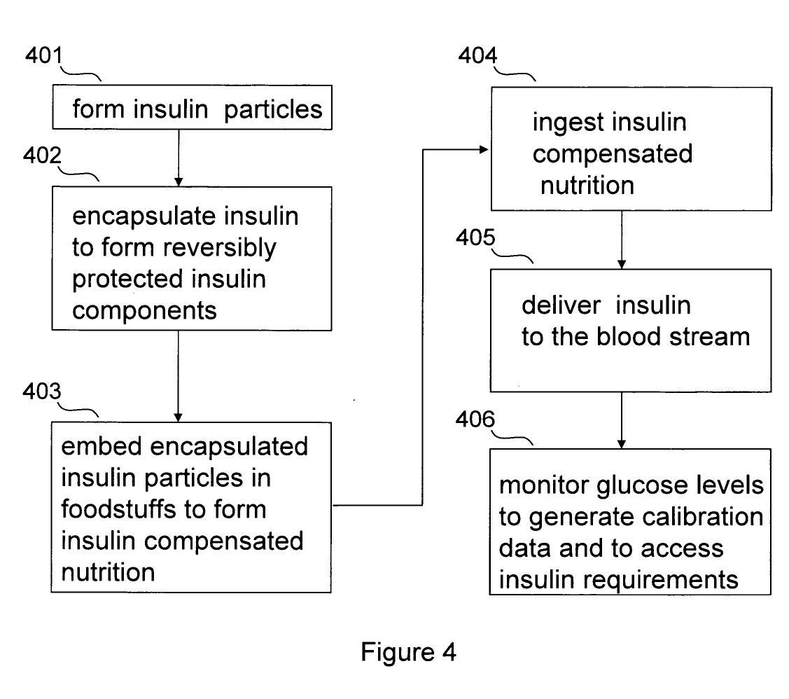

The inventive method useful in controlling blood glucose levels is further illustrated in FIG. 4 where the inventive steps are as follows:

insulin particles are formed 401 and encapsulated 402 to form reversibly protected insulin components which are then embedded or mixed with a foodstuff to form insulin compensated nutrition 403 which is then ingested 404. The reversibly protected insulin is delivered 405 to the blood stream by being released from the protective encapsulation in response to a pH level. The glucose level is monitored 406 by a glucose measuring device which generates glucose level data which can include an assessment of insulin requirements and monitor calibration data.

Insulin compensated nutrients can be used to compensate for the glucose content of other nutrients, such as conventional meals, by selecting insulin compensated nutrients with sufficient embedded insulin to substantially compensate for the total glucose content of all of the nutrients. Selecting insulin compensated nutrients can be based on known (published) glucose content of nutrients or can be based on previous experience of combining specific insulin compensated nutrients and other nutrients. Experienced based selection of insulin compensated nutrients may be enhanced by use of continuous of frequent glucose monitors.

Convenient use of insulin compensated nutrients can be enhanced by making multiple variations of embedded reversibly protected insulin containing capsules. Variations include, but are not limited to: varying the average concentration of the embedded insulin; varying the sensitivity of the protective coatings or capsules; varying the time to release and absorption into blood of the embedded insulin; varying the temporal profile of the release and absorption into blood of the embedded insulin. Variation of time to release may also be controlled by making insulin compensated nutrients available as drink as well as food nutrients.

Various instantiations of insulin compensated nutrients can be made by using various flavors, textures and compositions, etc. Such variations can be employed to make the insulin compensated nutrients more appetizing or palatable. Such variations can also be employed to readily identify insulin compensated nutrients with different magnitudes of excess insulin (used, for example, to compensate for additional nutrients).

It is understood that the above description is intended to be illustrative and not restrictive. Many of the features have functional equivalents that are intended to be included in the invention as being taught. For example, instead of pH sensitive hydro-gels, protective capsules that are sensitive to salinity, temperature, or antigens could be used. Reversibly protective mechanisms other than hydro-gels could be used, for example other molecular containing structures that are stable in a low pH environment but dissolve or change to release an insulin particle (or particles) in a higher pH environment.

Chewing gum type nutrient substances could be used to further control the timed release of reversibly protected insulin. The extent and duration of chewing could control the amount of reversibly protected insulin released for ingestion. Such chewing gum type nutrient could contain relative little glucose and be used to compensate for additional nutrients such as conventional food.

Specific insulin compensated nutrients could be designed to facilitate calibrating non-invasive glucose measuring system. Specific insulin compensated nutrients include, but are not limited to: nutrients that contain known quantities of embedded insulin in one form or another; nutrients that contain known quantities of glucose and known quantities of embedded insulin in one form or another; nutrients that contain known quantities of embedded insulin in one form or another with known timed release characteristics; and nutrients that contain known quantities of glucose and known quantities of embedded insulin in one form or another with known timed release characteristics.

Such specific insulin compensated nutrients would have predictable time dependent effects on glucose levels of various fluids, such as blood and interstitial and extra-cellular fluid. Such predictable time dependent effects on glucose levels would facilitate measuring glucose level changes, rates of change, time offsets between glucose level changes and rates of change which in turn facilitate determining absolute glucose information that can be processed and used generate insulin requirements (including insulin dosage levels and appropriate delivery time information) and also calibration data which can be used to calibrate glucose measurement systems. Such glucose measurement systems, include but are limited to, sporadic and continuous glucose measurement systems; invasive and non-invasive measurement systems. Such glucose measurement systems are herein referred to as glucose level monitoring devices.

Many variations and combinations of the above arrangements are possible, for example, hydro-gels could be encased in protective coatings that react to different environments or at different rates in similar environments. Multiple insulin particles could be encapsulated in single hydro-gels. Insulin particles contained within hydro-gels could have additional reversible protective coatings.

The digestive complex described in the preferred embodiment relates to the human digestive system. This invention also includes veterinary applications where the digestive complex may differ from the human digestive system.

Various forms of reversibly protected insulin components (other than hydro-gels) can be used. For example, reversibly protected insulin components include, but are not limited to nano-particles of insulin with a coating that is glucose responsive such that the permeability of the nano-particles varies with glucose concentration and thereby can remain in an environment where the insulin is not substantially absorbed or can move to an environment where it is more strongly absorbed depending on the glucose concentration level.

Other examples will be apparent to persons skilled in the art. The scope of this invention should be determined with reference to the specification, the drawings, the appended claims, along with the full scope of equivalents as applied thereto.

What is claimed is:

1. A method for generating a insulin compensated nutrient comprising:
generating a set of reversibly protected insulin components that substantially constitutes a compensating quantity of insulin; and
embedding said set of reversibly protected insulin components in said nutrient to form an insulin compensated nutrient.

2. The method of claim 1, wherein the protected insulin components are protected by being encapsulated in an acidic resistant capsule.

3. The method of claim 1, wherein the protected nature of the insulin components is reversible by exposure to an environment change.

4. The method of claim 3, wherein the environment change is a change in Ph value.

5. The method of claim 1, wherein the reversible aspect renders the protected insulin available for absorption into the blood stream.

6. The method of claim 1, wherein the compensating quantity of insulin compensates by providing an amount of insulin that substantially balances the amount of glucose in the nutrient.

7. The method of claim 1, wherein the compensating quantity of insulin compensates by providing an amount of insulin that substantially balances the amount of glucose delivered to the blood stream by the nutrient.

8. The method of claim 1, wherein the compensating quantity of insulin compensates by providing an amount of insulin that substantially balances the amount of glucose delivered to the blood stream by the insulin compensated nutrient and additional nutrients.

9. The method of claim 1, wherein the compensating quantity of insulin is determined by analyzing the glucose content of nutrients.

10. The method of claim 1, wherein the compensating quantity of insulin is determined by monitoring the glucose concentration response to similar nutrients.

11. An insulin compensated nutrient comprising:
a set of reversibly protected insulin components that substantially constitutes a compensating quantity of insulin; and
a nutrient, within which are embedded said set of reversibly protected insulin components so as to form an insulin compensated nutrient.

12. The insulin compensated nutrient of claim 11, wherein the protected insulin components are protected by being encapsulated in an acidic resistant capsule.

13. The insulin compensated nutrient of claim 11, wherein the protected nature of the insulin components is reversible by exposure to an environment change.

14. The insulin compensated nutrient of claim 13, wherein the environment change is a change in Ph value.

15. The insulin compensated nutrient of claim 11, wherein the reversible aspect renders the protected insulin available for absorption into the blood stream.

16. The insulin compensated nutrient of claim 11, wherein the compensating quantity of insulin compensates by providing an amount of insulin that substantially balances the amount of glucose in the nutrient.

17. The insulin compensated nutrient of claim 11, wherein the compensating quantity of insulin compensates by providing an amount of insulin that substantially balances the amount of glucose delivered to the blood stream by the nutrient.

18. The insulin compensated nutrient of claim 11, wherein the compensating quantity of insulin compensates by providing an amount of insulin that substantially balances the amount of glucose delivered to the blood stream by the insulin compensated nutrient and additional nutrients.

19. The insulin compensated nutrient of claim 11, wherein the compensating quantity of insulin is determined by analyzing the glucose content of nutrients.

20. The insulin compensated nutrient of claim 11, wherein the compensating quantity of insulin is determined by monitoring the glucose concentration response to similar nutrients.

21. A system for controlling glucose levels said system comprising:
an insulin compensated nutrient containing a set of reversibly protected insulin components; and
a digestive complex operable to deliver insulin from the insulin compensated nutrient to a blood stream, wherein the glucose level of said blood stream is controlled.

22. The system of claim 21, further including a glucose level monitoring device.

23. The system of claim 21, further including a glucose level monitoring device that generates calibration data.
